(19) United States
(12) Patent Application Publication
(10) Pub. No.: US 2023/0248895 A1
(43) Pub. Date: Aug. 10, 2023

(54) DEVICES FOR REMOVAL OF VISCERAL FAT, AND RELATED SYSTEMS AND METHODS

(71) Applicant: MEDALITY MEDICAL LLC, New York, NY (US)

(72) Inventors: Mark Andrew, Haddonfield, NJ (US); Richard Rogers, Hudson, MA (US)

(21) Appl. No.: 18/186,705

(22) Filed: Mar. 20, 2023

Related U.S. Application Data
(63) Continuation of application No. 17/196,475, filed on Mar. 9, 2021, now abandoned.
(60) Provisional application No. 62/988,222, filed on Mar. 11, 2020.

(51) Int. Cl. A61M 1/00

(52) U.S. Cl. CPC A61M 1/77; A61M 2202/0085; A61M 2202/08; A61M 2210/1017; A61M 2202/0014; A61M 2205/3368

(57) ABSTRACT

Methods of treating a medical condition in a patient include performing a lipectomy of visceral fat to remove a quantity of the visceral fat from the subject. The removal of visceral fat treats the medical condition such that the subject experiences at least a reduction of symptoms of the medical condition.

### CROSS REFERENCE TO RELATED APPLICATIONS

This application is a continuation of U.S. patent application Ser. No. 17/196,475, filed Mar. 9, 2021, and claims the benefit of U.S. Provisional Patent Application No. 62/988,222, filed Mar. 11, 2020, the entire disclosure of both of which are incorporated herein by this reference.

### TECHNICAL FIELD

The present invention relates to devices for removing visceral fat in vivo, as well as to systems and methods related to removing visceral fat in vivo.

### BACKGROUND

It has come to light in recent years that visceral fat, unlike subcutaneous fat, poses significant risks to general health. Visceral fat is now viewed as not just an inert blob of fat, but as another endocrine organ and, further, a “high risk” endocrine organ. For example, visceral fat secretes chemical mediators which are believed to have deleterious effects, including causing insulin resistance and creating a systemic inflammatory state in the body. Visceral fat therefore is now viewed not only as a culprit in the pathogenesis of diabetes mellitus type II but also is suspected as a possible contributor to hypertension, cardiovascular disease, cancer, obesity, Alzheimer's disease, dementia, aging itself, non-alcoholic fatty liver disease (NAFLD), and non-alcoholic steatohepatitis (NASH), among other diseases and disorders.

Liposuction, also known as lipoplasty (fat modeling), liposculpture, or suction lipectomy (suction-assisted fat removal) is a cosmetic surgery operation that removes subcutaneous fat from many different sites on the human body (e.g., the chest, buttocks, hips, thighs, or arms). The typical liposuction procedure relies on the action of a sharp-edged instrument to shear away the fatty deposits. The sheared fatty deposits are then suctioned away into orifices on the cannula. This process is labor-intensive for the surgeon, traumatic to non-fat tissues and to the patient, and very time consuming.

Typical liposuction tools and methods cannot be used for visceral fat lipectomy in the same manner as they are used to remove subcutaneous fat because visceral fat contains, among other things, a plethora of delicate blood vessels, nerves and lymphatic vessels and is attached to sensitive internal organs. Those vital, non-fat tissues and organs will not tolerate the repeated thrusting and shearing required with traditional lipectomy.

### SUMMARY

According to an embodiment of the present disclosure, methods of removing visceral fat from a subject include inserting at least a distal portion of a cannula into the anatomy of the subject, wherein the anatomy comprises peritoneal cavity, the retroperitoneal cavity, or visceral fat tissue that has been moved from its natural anatomical location in the body. The cannula is elongate along a longitudinal direction. The distal portion of the cannula defines at least one aperture that is open to an interior cavity of the cannula. The methods can include generating a negative pressure in the interior of the cannula so that the negative pressure draws a portion of visceral fat from the peritoneal cavity or the retroperitoneal cavity through the at least one aperture and into the interior cavity. The methods can further include delivering fluid through a conduit of the cannula along the longitudinal direction in a series of pulses that are individually at a pressure in a range of 600 psi to 1300 psi and at a temperature in a range of 80 degrees Fahrenheit and 140 degrees Fahrenheit. Representative methods include expelling the fluid in the series of pulses from the conduit, impacting the expelled fluid against the portion of visceral fat so as to liquefy the visceral fat, and suctioning at least a major portion of the liquefied visceral fat through the interior of the cannula and away from the subject responsive to the negative pressure.

According to another embodiment of the present disclosure, methods of treating a medical condition in a patient include performing a lipectomy of visceral fat to remove a quantity of the visceral fat from the subject. The removal of visceral fat treats the medical condition such that the subject experiences at least a reduction of symptoms of the medical condition.

According to an additional embodiment of the present disclosure, a cannula for removing visceral fat from a subject in vivo includes a cannula body defining a proximal end and a distal end spaced from each other along a longitudinal direction. The cannula body also defines an interior cavity and at least one aperture that is open to the interior cavity and is configured to draw visceral fat into the interior cavity responsive to vacuum pressure supplied to the interior cavity. The at least one aperture is located adjacent the distal end and remote from the proximal end. The cannula includes at least one fluid supply tube that extends along the longitudinal direction within the interior cavity and has a nozzle at a terminal end thereof. The fluid supply tube is configured to deliver fluid toward the aperture in a series of pulses that are individually at a pressure in a range of 600 psi to 1300 psi and at a temperature in a range of 80 to 140 degrees Fahrenheit. The nozzle is configured to eject the series of boluses from the at least one fluid supply tube and against visceral fat drawn into the interior cavity. The cannula can include at least one cauterizing electrode adjacent the distal end.

According to yet another embodiment of the present disclosure, a cannula for removing visceral fat from a subject in vivo includes a cannula body defining a proximal end and a distal end spaced from each other along a longitudinal direction. The cannula body also defining an interior cavity and at least one aperture that is open to the interior cavity and is configured to draw visceral fat into the interior cavity responsive to vacuum pressure supplied to the interior cavity. The at least one aperture is located adjacent the distal end and remote from the proximal end. The cannula includes at least one fluid supply tube that extends along the longitudinal direction within the interior cavity and has a nozzle at a terminal end thereof. The fluid supply tube is configured to deliver fluid toward the aperture in a series of pulses that are individually at a pressure in a range of 600 psi to 1300 psi and at a temperature in a range of 80 to 140 degrees Fahrenheit. The nozzle is configured to eject the series of boluses from the at least one fluid supply tube and against visceral fat drawn into the interior cavity. The cannula includes an outer sleeve that is positioned over the outer surface of the cannula body. The outer sleeve defines at least one sleeve aperture aligned with the at least one aperture of the cannula.

According to a further embodiment of the present disclosure, a cannula for removing visceral fat from a subject in vivo includes a cannula body defining a proximal end and a distal end spaced from each other along a longitudinal direction. The cannula body defining an interior cavity and first and second apertures that are open to the interior cavity and are each configured to draw visceral fat into the interior cavity responsive to vacuum pressure supplied to the interior cavity. The first and second apertures are adjacent the distal end and remote from the proximal end. The first and second apertures are also spaced from each other along a circumference of the cannula body. The cannula includes at least one fluid supply tube that extends along the longitudinal direction within the interior cavity and is configured to deliver fluid toward one or both of the first and second apertures in a series of pulses that are individually at a pressure in a range of 600 psi to 1300 psi and at a temperature in a range of 80 to 140 degrees Fahrenheit. The cannula includes at least one nozzle at a terminal end of the at least one fluid supply tube. The at least one nozzle is configured to eject and the series of boluses from the at least one fluid supply tube and impact the series of boluses against visceral fat drawn into the interior cavity.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the structures of the present application, there is shown in the drawings illustrative embodiments. It should be understood, however, that the application is not limited to the precise arrangements and instrumentalities shown. In the drawings:

FIG. 1 is a diagrammatic view of a tissue liquefaction system that includes a lipectomy cannula, according to an embodiment of the present disclosure;

FIG. 2 is a bottom plan view of a distal portion of the lipectomy cannula illustrated in FIG. 1, according to an embodiment of the present disclosure;

FIG. 3 is a sectional side view of an alternative configuration of the distal portion of the cannula, as if taken along section line 3-3 illustrated in FIG. 2, according to another embodiment of the present disclosure;

FIG. 4 is a bottom plan view of a distal portion of the lipectomy cannula, according to another embodiment of the present disclosure;

FIG. 5 is a block diagram of a fluid heating and pressurization system, according to another embodiment of the present disclosure;

FIG. 6 is a sectional side view of a distal portion of a cannula having a cauterizing electrode and an outer sleeve, according to another embodiment of the present disclosure;

FIG. 7 is a sectional side view of a distal portion of a cannula having a cauterizing electrode and an outer sleeve, according to another embodiment of the present disclosure;

FIG. 8 is a bottom plan view of the distal portion of the cannula illustrated in FIG. 6;

FIGS. 9A-9E are each bottom plan views of the distal portion of a cannula having multiple side-by-side apertures, including two (2) side-by-side apertures (FIGS. 9A-9D) and three (3) side-by-side apertures (FIG. 9E), according to additional embodiments of the present disclosure;

FIG. 10A is a sectional side view of a cannula having a bend, according to another embodiment of the present disclosure;

FIG. 10B shows an alternative cannula configuration with multiple bends;

FIG. 11 is a diagram view of surgical system for performing laparoscopic removal of visceral fat, according to another embodiment of the present disclosure; and

FIG. 12 is a plan view of a positioning tool for components of the surgical system illustrated in FIG. 11.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

As used herein with reference to fat (adipose) tissue, the terms “liquefy”, “liquefaction”, and their derivatives include actions of liquefying, softening, gellifying, and/or causing adipocyte cell disaggregation of the fat tissue.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately” and “substantially”, as used herein with respect to dimensions, angles, and other geometries, takes into account manufacturing tolerances. Further, the terms “approximately” and “substantially” can include 10% greater than or less than the stated dimension or angle. Further, the terms “approximately” and “substantially” can equally apply to the specific value stated.

The embodiments described below generally involve the delivery of pressurized heated biocompatible fluid to heat targeted tissue and soften, gellify, or liquefy the target tissue for removal from a living body. The heated biocompatible fluid is preferably delivered as a series of pulses, but in alternative embodiments can be delivered as a continuous stream. After the tissue has been softened, gellified, or liquefied, it is sucked away out of the subject's body.

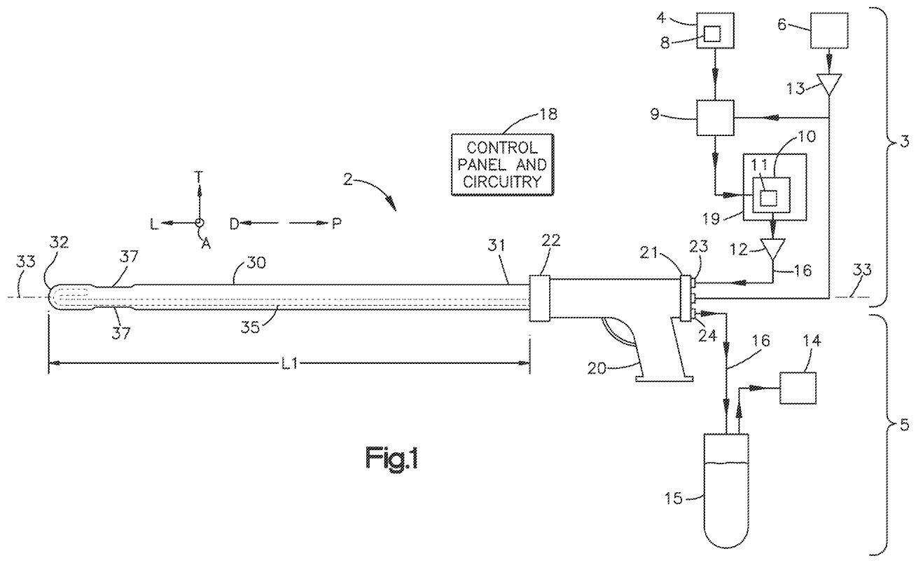

Referring now to FIGS. 1-4, a tissue liquefaction system 2 includes a lipectomy cannula 30 having a distal end 32 that is smooth and rounded for introduction into the subject's body. A proximal end 31 of the cannula 30 is configured to mate with a handpiece 20. The cannula 30 is elongate along a longitudinal direction L and defines a longitudinal axis 33 oriented along the longitudinal direction L. The distal end 32 is spaced from the proximal end 31 in a distal direction D. Moreover, the proximal end 31 is spaced from the distal end 32 in a proximal direction P. It should be appreciated that the distal and proximal directions D, P are each mono-directional components of the longitudinal direction L, which is bi-directional. The cannula 30 defines an interior cavity 90 and one or more apertures 37 that are open to the cavity 90. These apertures 37 are in fluid communication with the interior cavity 90 and are preferably located near a distal portion of the cannula 30. When a negative pressure (i.e., vacuum pressure) source is connected to the cavity 90 via a suitable fitting, suction can be generated which draws target visceral fatty tissue into the apertures 37. Accordingly, the apertures 37 can also be referred to as “suction apertures” 37 or “induction apertures” 37.

The one or more apertures 37 are open to the cavity 90 along a transverse direction T that is substantially perpendicular to the longitudinal direction L. Additionally, the one or more apertures 37 each define an aperture shape, which can be characterized with respect to a reference plane, such as a reference plane extending along the longitudinal direction L and a lateral direction A. For purposes of this disclosure, the longitudinal, lateral, and transverse directions L, A, T are substantially perpendicular to each other and effectively define a three-axis coordinate system in three-dimensional space.

The cannula 30 also includes one or more fluid supply tubes 35 that direct the heated fluid onto the target tissue that has been drawn into the cavity 90. These fluid supply tubes 35 are preferably arranged within the interior cavity 90, although in other embodiments, proximal portions of the fluid supply tubes 35 can extend externally from the cannula 30, as more fully described in U.S. Pat. No. 8,366,700, issued Feb. 5, 2013, entitled “LIPOSUCTION OF VISCERAL FAT USING TISSUE LIQUEFACTION” (hereinafter referred to as “the '700 Reference”), the entire disclosure of which is incorporated by reference herein. The fluid supply tubes 35 are arranged to eject the fluid across interior aspects of the apertures 37, respectively, so that the fluid impacts against the target fatty tissue that has been drawn into the cavity 90.

The tissue liquefaction system 2 includes a fluid delivery sub-system 3 that includes a fluid supply reservoir 4, a heat source 8 that heats the fluid in the reservoir 4, and a temperature regulator 9 that controls the heat source 8 as required to maintain the desired temperature. The heated fluid from the fluid supply 4 is delivered under pressure by a suitable arrangement 10 such as a pump system 19 with a pressure regulator 11. Optionally, a heated fluid metering device 12 can also be provided to measure the fluid that has been delivered.

The fluid delivery sub-system 3 includes a pump 19 that pumps the heated fluid from the reservoir or fluid supply source 4 down the fluid supply tubes 35 that run from the proximal end 31 of the cannula 30 down to the distal end 32 of the cannula 30. Near the distal tip 32 of the cannula 30, these fluid supply tubes 35 can optionally make a U-turn so as to face back towards the proximal end of the cannula 30. As a result, when the heated fluid exits such supply tubes 35 at their respective delivery orifices 43 (also referred to herein as “fluid ejection nozzles” 43 or simply “nozzles” 43), the fluid is traveling substantially in the proximal direction P. Preferably, the pump 19 delivers a pressurized, pulsating output of heated fluid down the supply tube 35 so that a series of boluses of fluid are ejected from the nozzle 43, as described in greater detail below.

The tissue liquefaction system 2 includes a suction subsystem 5 that includes a vacuum source 14. The fluid delivery sub-system 3 and the suction sub-system can interface with the cannula 30 via a handpiece 20. The heated solution supply is connected on the proximal side of hand piece 20 with a suitable fitting, and a vacuum supply is also connected to the proximal side of handpiece 20 with a suitable fitting. Cannula 30 is connected to the distal side of hand piece 20 with suitable fittings so that (a) the heated fluid from the fluid supply is routed to the supply tubes 35 in the cannula and (b) the vacuum is routed from the vacuum source 14 to the cavity 90 in the cannula 30, to evacuate material from the cavity 90.

More specifically, the pressurized heated solution that is discharged from pump 19 is connected to the proximal end of the handle 20 via flexible tubing (capable of delivering pressurized fluid), and routed through the handpiece 20 to the cannula 30 with an interface made using an appropriate fitting. The vacuum source 14 is connected to an aspiration collection canister 15, which in turn is connected to the proximal end of the handle via flexible tubing 16, and then routed through the handpiece 20 to the cannula 30 with an interface made using an appropriate fitting. The pressurized fluid supply line connection between the handle and the cannula 30 can be implemented using a quick disconnect fitting located at the distal end of the handle, and configured so that once the cannula is inserted into the distal end of the handle it aligns and connects with both the fluid supply and the vacuum supply. The cannula 30 can be held in place on the handle 20 by an attachment cap.

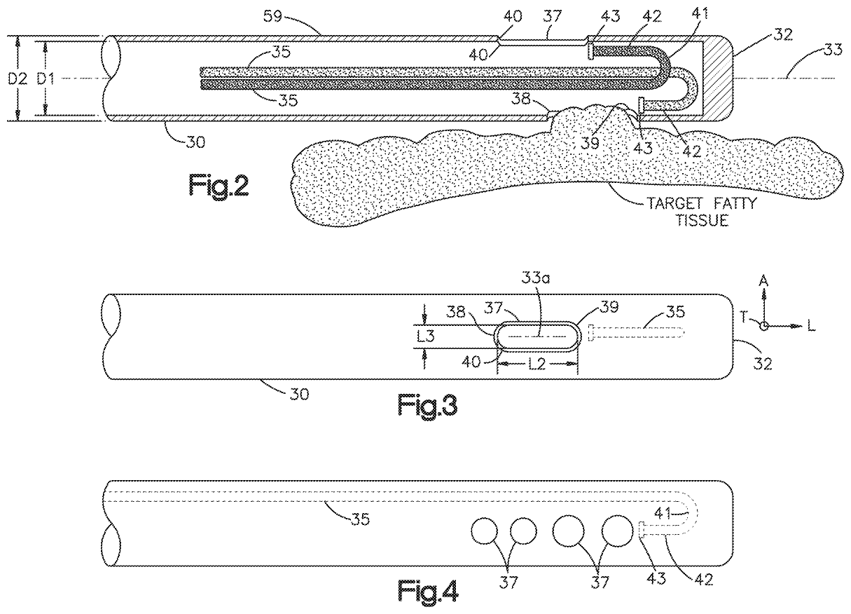

Referring now to FIG. 2, after the cannula 30 is inserted into the fat tissue; vacuum source 14 creates a vacuum pressure region within the cavity 90 of the cannula 30 such that the target fatty tissue is drawn into the cannula 30 through the aperture 37. The geometry of the end of the supply tube 35 is configured so the trajectory of the boluses leaving the nozzle 43 will strike the fatty tissue that has been drawn into the cannula 30 through the aperture 37. For that purpose, the nozzle 43 at the end of the supply tube 35 preferably points in a direction that is substantially parallel to that of the inside wall of the cannula 30 where the supply tube 35 is affixed. Preferably, the supply tube 35 and nozzle 43 are oriented so that the stream flows across the aperture 37 in the proximal direction P. This placement of the nozzle 43 of the supply tube 35 advantageously maximizes the energy transfer (kinetic and thermal) to the fatty tissues, minimizes fluid loss, and helps prevent clogs by pushing the heated fluid and the liquefied material in the same direction that it is being pulled by the vacuum source.

Once the targeted fatty tissue enters the aperture 37, it is repeatedly impacted by the boluses of heated fluid that are exiting the supply tubes 35 via the nozzle 43. Adipocytes, which comprise the majority of the targeted fatty tissue mass, undergo a virtually instantaneous process of partial cell disaggregation caused by the impacting boluses of fluid and the fatty tissue is resultantly liquefied. The partial cell disaggregation produces a lipoaspirate that is a multi-cell suspension composed of tiny clusters of fat cells. Such a multi-cell suspension is not a single cell suspension composed of individual fat cells, although it should be appreciated that the multi-cell suspension can include a minor amount of individual fat cells in the suspension. After the partial cell disaggregation occurs, the loose material in the cavity 90 (i.e., the heated fluid and the liquefied fat tissue) is drawn away from the surrounding tissue by the vacuum source 14, and deposited into the canister 15 (shown in FIG. 1).

In the present embodiment, the proximal direction P of the boluses is the same as the direction that the liquefied tissue travels when it is being suctioned out of the patient via the cannula 30. By having the fluid stream flow in the proximal direction P, additional energy (vacuum, fluid thermal and kinetic) is transferred in the same direction, which aids in moving the aspirated tissues through the cannula 30. This further contributes to reducing clogs, which can reduce the time it takes to perform a procedure.

Notably, in the embodiments described herein, the majority of the fluid stays within the interior of the cannula during operation (although a small amount of fluid can escape into the subject's body through the aperture 37). This is advantageous because minimizing fluid leakage from the cannula 30 maximizes the energy transfer (thermal and kinetic) from the fluid stream to the tissue drawn into the cannula 30 for liquefaction.

Each of the supply tubes 35 is provided for delivering the heated fluid. Supply tube 35 extends from the proximal portion of cannula 30 toward the distal tip 32 of cannula 30. Supply tube 35 extends along the interior of cannula 35 and can be a separate structure secured to the interior of cannula 35 or lumen integrated into the wall of cannula 30. Supply tube 35 is configured to deliver heated biocompatible solution for liquefying tissue. The heated solution is delivered through hand piece 20 and into supply tube 35.

The supply tube 35 extends longitudinally (i.e., along the longitudinal direction L) from the proximal end 31 to the distal tip 32. Supply tube 35 can optionally include a bend, such as a U-bend 41 that effectively redirects the direction of fluid flow to the proximal direction P. Adjacent the terminal end of the U-bend 41 is a supply tube terminal portion 42, which includes the nozzle 43. The nozzle 43 is configured to direct heated solution exiting supply tube 35 across the aperture 37. In this manner, supply tube 35 is configured to direct the fluid onto a target tissue that has entered the cannula 30 through the aperture 37. As an alternative to the U-bend 41 configuration, the supply tube 35 can include a manifold that is located at the distal portion of the cannula 30 and includes a nozzle 43 for each aperture 37.

Heated solution supply tube 35 can be constructed of surgical grade tubing. Alternatively, in embodiments wherein the heated solution supply tube is integral to the construction of cannula 30, the supply tube 35 can be made of the same material as cannula 30. The diameter of supply tube 35 can be dependent on the target tissue volume requirements for the heated solution and on the number of supply tubes 35 required to deliver the heated solution across the one or more apertures 37. The cannulas 30 described herein define inner diameters D1 or “tube” diameters that can vary with respective outer diameters D2 of the cannulas 30. The fluid supply tube 35 defines an inner diameter, which can be in a range from about 0.008 inch. to 0.032 inch. In one preferred embodiment, the portion of the supply tube 35 within the cannula 30 has an inner diameter of about 0.02 inch and an exit nozzle 43 formed by reducing the inner diameter to about 0.013 inch along the final 0.1 inch of length of the supply tube 35. The shape and size of the nozzle 43 can vary, including reduced diameter and flattened configurations.

In other embodiments, the cannula 30 can have a different number of heated solution supply tubes 35, each corresponding to a respective aperture 37. For example, a cannula 30 with three apertures 37 would preferably include three heated solution supply tubes 35. Additionally, heated solution supply tubes can be added to accommodate one or more induction ports, e.g., when four apertures 37 are provided, four heated solution supply tubes can be provided. In another embodiment, a supply tube 35 can branch into multiple tubes, each branch servicing a induction port. In another embodiment, one or more supply tubes can deliver the heated fluid to a single induction port. In yet another embodiment, supply tube 35 can be configured to receive one or more fluids in the proximal portion of cannula 30 and deliver the one or more fluids though a single nozzle 43. In another embodiment, the cannula 30 can be attached to an endoscope or other imaging device.

The heated fluid should be biocompatible, and can comprise a sterile physiological serum, normal saline solution, glucose solution, Ringer-lactate, hydroxyl-ethyl-starch, or a mixture of these solutions. The heated biocompatible fluid can also comprise saline or sterile water or can be comprised solely of saline or sterile water.

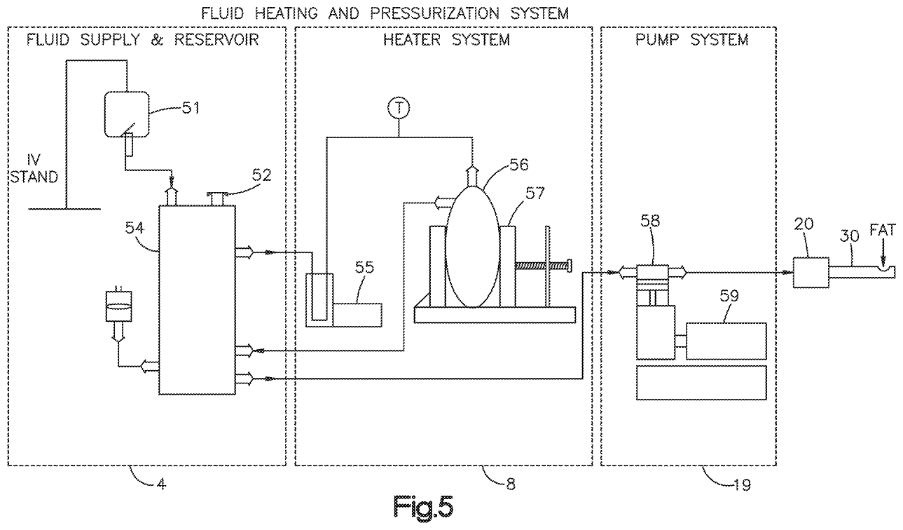

Referring now to FIG. 5, another embodiment of a fluid delivery sub-system 3 for heating and delivering the fluid to the cannula 30 will be described. The components in FIG. 5 can operate using the following steps: Room temperature saline drains from the IV bag 51 into mixing storage reservoir 54. Once the fluid in the reservoir 54 reaches a fixed limit, the fixed speed peristaltic pump 55 of the heater system 8 moves fluid from the reservoir 54 to the heater bladder 56. The fluid is circulated through the bladder and is heated by the electric panels 57 of the heater system 8. The heated fluid is returned back to the reservoir 54 and mixes with the other fluid in the storage container. The fixed speed peristaltic pump 55 continues to circulate fluid to the heater unit and back into the reservoir 54. The continuous circulation of fluid provides a very stable and uniform heated fluid volume supply. Temperature control can be implemented using any conventional technique, which will be readily apparent to persons skilled in the relevant arts, such as a thermostat or a temperature-sensing integrated circuit. The temperature can be set to a desired level by any suitable user interface, such as a dial or a digital control, the design of which will also be apparent to persons skilled in the relevant arts.

The pump 58 can be a piston-type, positive displacement pump that draws heated fluid from the fluid reservoir 54 into the pump chamber when the pump plunger travels in a backstroke. The fluid inlet to the pump has an in-line one-way check valve that allows fluid to be suctioned into the pump chamber, but will not allow fluid to flow out. Once the pump plunger backstroke is completed, the forward travel of the plunger starts to pressurize the fluid in the pump chamber. The pressure increase causes the one-way check valve at the inlet of the pump 58 to shut preventing flow from going out the pump inlet. As the pump plunger continues its forward travel the fluid in the pump chamber increases in pressure. Once the pressure reaches the preset pressure on the pump discharge pressure regulator the discharge valve opens. This creates a bolus of pressurized heated fluid that travels from the pump 58 through cannula handle 20 and from there into the supply tube 35 in the cannula 30. After the pump plunger has completed its forward travel the fluid pressure decreases and the discharge valve shuts. These steps are then repeated to generate a series of boluses. Suitable repetition rates (i.e., pulse rates) are discussed below.

One example of a suitable approach for implementing the positive displacement pump is to use an off-set cam on the pump motor that causes the pump shaft to travel in a linear motion. The pump shaft is loaded with an internal spring that maintains constant tension against the off-set cam. When the pump shaft travels backwards towards the off-set cam it creates a vacuum in the pump chamber and suctions heated saline from the heated fluid reservoir. A one-way check valve is located at the inlet port to the pump chamber, which allows fluid to flow into the chamber on the backstroke and shuts once the fluid is pressurized on the forward stroke. Multiple inlet ports can allow for either heated or cooled solutions to be used. Once the heated fluid has filled the pump chamber at the end of the pump shaft backwards travel, the off-set portion of the cam will start to push the pump shaft forward. The heated fluid is pressurized to a preset pressure (e.g., 1100 psi) in the pump chamber, which causes the valve on the discharge port to open, discharging the pressurized contents of the pump chamber to fluid supply tubes 35. Once the pump plunger completes its full stroke based on the off-set of the cam, the pressure in the pump chamber decreases and the discharge valve closes. As the cam continues to turn the process is repeated.

The heated biocompatible solution in a tissue liquefaction system is preferably delivered in a manner optimized for liquefying the target tissue. Variable parameters include, without limitation, the temperature of the solution, the pressure of the solution, the pulse rate or frequency of the solution, and the duty cycle of the pulses or boluses within a stream, the rise rate (i.e., the speed with which the fluid is brought to the desired pressure) of the pulse, and the size of the bolus, which is determined by the specific parameters of the pump dimensions. Additionally, the vacuum pressure applied to the cannula through the vacuum source 14 can be optimized for the target tissue.

It has been found that for liposuction procedures targeting subcutaneous fatty deposits within the human body, the biocompatible heated solution should preferably be delivered to the target fatty tissue at a temperature between 75 and 250 degrees F., and more preferably between 100 and 140 degrees F. A particular preferred operating temperature for the heated solution is about 115 degrees F., since this temperature appears very effective and safe. Also, for liquefaction of fatty deposits the pressure of the heated solution is preferably between about 200 and about 2500 psi, more preferably between about 600 and about 1300 psi, and still more preferably between about 800 and about 1100 psi. A particular preferred operating pressure is about 900 psi, which provides the desired kinetic energy while minimizing fluid flow. The volume of each bolus (pulse) delivered to each aperture is preferably between about 200 microliters and about 275 microliters, more preferably between about 215 microliters and about 245 microliters, and still more preferably about 230-microliters. The pulse rate of the solution is preferably between 20 and 150 pulses per second, more preferably between 25 and 60 pulses per second. In some embodiments, a pulse rate of about 40 pulses per second was used. And the heated solution can have a duty cycle (i.e., the duration of the pulses divided by the period at which the pulses are delivered) of between 1-100%. In preferred embodiments, the duty cycle can range between 30 and 60%, and more particularly between 30 and 50%.

In preferred embodiments, the rise rate is about 0.1 to 3.0 milliseconds. This can be accomplished by having a standard relief valve that opens once the pressure in the pump chamber reaches the set point (which, for example, can be set to 1100 psi). The pressure increase is almost instantaneous and the fluid exits the fluid supply tube(s) 35 during a very short time span, as more fully described in the '700 Reference.

Returning now to the suction subsystem 5, FIG. 2 depicts an expanded cut-away view of an embodiment of the cannula 30 that includes two (2) apertures 37. As shown, the cannula 30 has two apertures 37 located near the distal region of the cannula 30 and proximal to distal tip 32. The apertures 37 can be positioned in various configurations about the perimeter of the distal region of the cannula 30. In the illustrated embodiment, the apertures 37 are on opposite sides of the cannula 30, but in alternative embodiments they can be positioned differently with respect to each other. The apertures 37 are configured to allow fatty tissue to enter the orifices in response to vacuum pressure within the cannula cavity 90 created by the vacuum supply 14. The material that is located in the cavity 90 (i.e., tissue that has been liquefied and the heated fluid that exited the supply tube 35) is then suctioned away in the proximal direction P up through the cannula 30, the handpiece 20, and into the canister 15 (all shown in FIG. 1). A conventional vacuum pump (e.g., the AP-III HK Aspiration Pump from HK Surgical Inc. of San Clemente, Calif.) can be used for the vacuum source 14, although other types of vacuum pumps can be employed.

In preferred embodiments, the aspiration vacuum that sucks the liquefied tissue (or at least a major portion thereof) back up through the cannula 30 ranges from 0.33-1.00 atmosphere (1 atmosphere=760 mm Hg). Optionally, the vacuum level can be adjustable by the surgeon during the procedure as needed. Because reduced aspiration vacuum is associated with lower blood loss, the surgeon can prefer to work at the lower end of the vacuum range.

Returning to FIGS. 1-4, the cannula 30 and handpiece 20 will now be described in greater detail. Hand piece 20 has a proximal end 21 and a distal end 22, a fluid supply connection 23 and a vacuum supply connection 24 preferably located at the proximal end, and a fluid supply fitting and a vacuum supply fitting at the distal end (to interface with the cannula). The hand piece 20 routes the heated fluid from the fluid supply to the supply tubes 35 in the cannula 30 and routes the vacuum from the vacuum source 14 to the cavity 90 in the cannula 30, to evacuate material from the cavity 90. The handpiece 20 can have pistol-type configuration, as shown, although in preferred embodiments the handpiece 20 has a wand-type configuration.

In some embodiments, a cooling fluid supply 6 can be used to dampen the heat effect of the heated fluid stream in the surgical field. In these embodiments, the handpiece also routes the cooling fluid into the cannula 35 using appropriate fittings at each end of the handpiece. In these embodiments, a cooling fluid metering device 13 can optionally be included. The hand piece 20 can optionally include operational and ergonomic features such as a molded grip, vacuum supply on/off control, heat source on/off control, alternate cooling fluid on/off control, metering device on/off control, and fluid pressure control. Hand piece 20 can also optionally include operational indicators including cannula aperture 37 location indicators, temperature and pressure indicators, as well as indicators for delivered fluid volume, aspirated fluid volume, and volume of tissue removed. Alternatively, one or more of the aforementioned controls can be placed on a separate control panel 18.

The distal end 22 of hand piece 20 is configured to mate with the cannula 30. The cannula 30 comprises a hollow tube of surgical grade material, such as stainless steel, that extends from a proximal end 31 and terminates in a rounded tip at a distal end 32. The proximal end 31 of the cannula 30 attaches to the distal end 22 of hand piece 20. Attachment can be by means of threaded screw fittings, snap fittings, quick-release fittings, frictional fittings, or any other attachment connection known in the art. It will be appreciated that the attachment connection should prevent dislocation of cannula 30 from hand piece 20 during use, and in particular should prevent unnecessary movement between cannula 30 and hand piece 20 as the surgeon moves the cannula hand piece assembly in a back and forth motion approximately parallel to the cannula longitudinal axis 33.

The cannula 30 can include designs of various diameters, lengths, curvatures, and angulations to allow the surgeon anatomic accuracy based upon the part of the body being treated, the amount of fat extracted as well as the overall patient shape and morphology. This would include cannula 30 outer diameters D2 of 2.0 mm for delicate precise liposuction of small fatty deposits of about 1 mm in size to cannulas 30 with outer diameters D2 up to 7.0 mm for large volume fat removal (i.e., abdomen, buttocks, hips, back, thighs etc.), and cannula 30 lengths L1 from 2 cm for small areas (i.e., eyelids, cheeks, jowls, face etc.) up to 50 cm in length for larger areas and areas on the extremities (i.e., legs, arms, calves, back, abdomen, buttocks, thighs, etc.). A myriad of cannula 30 designs include, without limitation, a C-shaped curves of the distal tip alone, S-shaped curves, step-off curves from the proximal or distal end as well as other linear and nonlinear designs. The cannula 30 can be a solid cylindrical tube, articulated, or flexible.

Each of the apertures 37 includes a proximal end 38, a distal end 39, and a perimeter edge 40. Although the illustrated apertures 37 are oval, round, or oblong, in alternative embodiments they can be made in other shapes (e.g., egg shaped, diamond or polygonal shaped, or an amorphous shape). As depicted in FIG. 3, the apertures 37 can be arranged in a longitudinally elongated fashion on one or more sides of the cannula 30. Alternatively, the apertures 37 can be provided in a series along the longitudinal direction L, as depicted in FIG. 4. Optionally, the dimensions or shape of each aperture 37 can change, for example, from the most distal aperture 37 to the most proximal aperture 37, as illustrated in FIG. 4, where the diameter of each aperture 37 can decrease in succession from the most distal aperture 37 to the most proximal aperture 37.

The perimeter edge 40 is configured to present a smooth, unsharpened edge to substantially eliminate shearing, tearing or cutting of the target fatty tissue and also to substantially avoid damage to adjacent non-target tissue caused by shearing, tearing or cutting, particularly for visceral fat removal. Because the target tissue is liquefied, the cannula 30 does not need to shear tissue.

For targeting visceral fat, the cannula 30 preferably has between one (1) and three (3) apertures 37, although more than three (3) apertures 37 can optionally be employed. The apertures 37 can have different shapes, such as round or oblong, by way of non-limiting examples. When oblong apertures 37 are used, a longitudinal axis 33a of the aperture 37 should preferably be oriented substantially along the longitudinal direction L. Accordingly, the oblong aperture 37 should have a longitudinal dimension L2 (i.e., length) that is greater that a lateral dimension L3 (i.e., width) thereof. Stated differently, the aperture 37 is preferably elongated along the longitudinal direction L. The apertures 37 should not be too large, because with smaller apertures 37 less fat is suctioned into the cannula 30 for a given bolus of energy. On the other hand they should not be too small, to permit the fatty tissue to enter. A suitable size for oblong apertures 37, according to one example embodiment, is a width L3 of about 0.04 inch and a length L2 of about 0.24 inch (i.e., about 0.04 inch wide×0.24 inch long). In further embodiments, the apertures 37 can be wider than 0.04 inch and longer than 0.24 inch. The size of the apertures 37 can further be varied for different applications depending on the surgeon's requirements. More extensive areas to be suctioned can require larger apertures 37.

It should be appreciated that the interior cavity 90 of the cannula 30 defines a suction path for the removal of visceral fat from the subject's body. The suction path 90 of the embodiments described herein can define a resistance ratio and a surface area per unit length of the suction path as more fully described in the '700 Reference.

Because visceral fat is located on and around organs that are more delicate and more vital than the anatomical structures in which subcutaneous fat is found (skin on one side, muscle on the other side), care must be taken to minimize trauma to the relevant anatomical regions, to permit removal the visceral fat without causing damage or trauma to the adjacent internal organs or to the plethora of blood and lymphatic vessels, and nerves that course throughout the mesenteric fat which supply the bowel. For example, the apertures 37 need to have perimeter edges 40 that are rounded, dull, or otherwise blunt so that they present non-cutting surfaces to target and non-target tissue, so as to avoid shearing and cutting of tissue. Additionally, the pressure and temperature ranges should be selected to avoid trauma to non-fat tissue during visceral fat removal.

The cannula 30 defines a cannula body 59 that is preferably constructed from cylindrical, elliptical, or flat face tubing, with no exaggerated blunt faces. Suitable materials for the cannula body 59 include surgical grade metallic materials like 304 and 316 stainless steel, as well as shape memory and super elastic metallic materials like Nitinol. The cannula 30 can also be made of non-metallic materials like polyetheretherketon (PEEK), polycarbonate (PC), high-density polyethylene (HDPE), nylon, and other high durometer plastics.

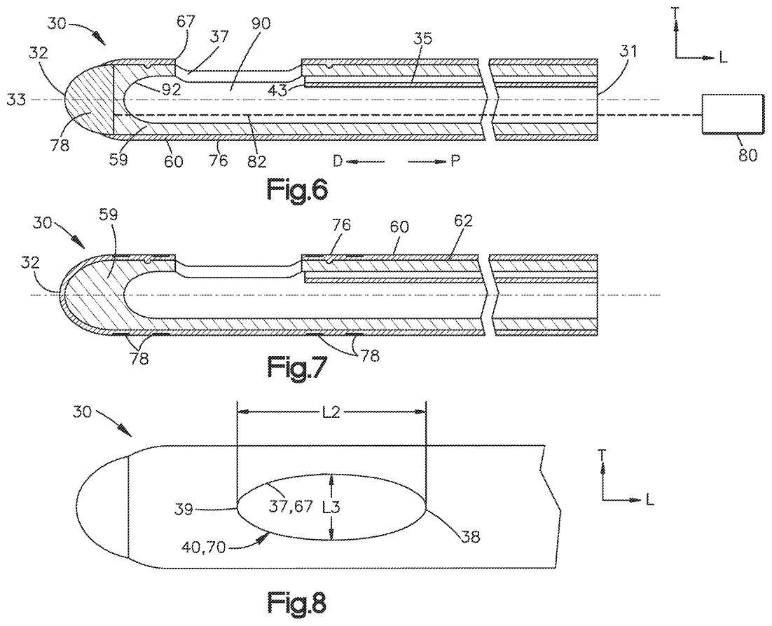

Referring now to FIG. 6, in additional embodiments, one or more of the fluid supply tubes 35 can have a nozzle 43 that ejects the series of fluid boluses in the distal direction D. In such embodiments, the cannula 30 can include a cannula body 59 that defines the interior cavity 90 and can further define therein a distal end surface 92, which can be configured to re-direct the loose fat tissue in the proximal direction P and toward the proximal end 31 of the cannula 30. The inventor has observed that the present embodiment, which directs the series of fluid boluses in the distal direction D to impinge against fatty tissue drawn into the interior cavity 90, exhibits favorable performance in removing fatty tissue. Furthermore, by locating each nozzle 43 proximally of the respective aperture 37, the need for a manifold (to redirect the fluid delivery tubes 35 to deliver the boluses in the proximal direction) can be obviated, which can provides a simplified manufacturing process and thereby reduce manufacturing costs. It is also believed that, by locating each nozzle 43 proximally of the respective aperture 37 for distal fluid ejection therefrom, the boluses can have a less turbulent, cleaner presentation to the fat, which is believed to enhance the rate of fat extraction.

The cannula 30 can also include an outer sleeve 60 disposed around an outer surface 62 of the cannula body 59. As shown, the outer sleeve 60 is preferably a continuous sheath surrounding the cannula body 59 from a sleeve proximal end 64 located at or adjacent the proximal end 31 of the cannula 30 to a sleeve distal end 66 located proximate the distal end 32 of the cannula 30. In other embodiments, the sleeve 60 can be braided or coiled around the outer surface 62 of the cannula body 59. The sleeve 60 defines at least one aperture 67 that is configured to align with at least one aperture 37 of the cannula 30. The aperture 67 of the sleeve 60 defines a proximal end 68, a distal and 69, and a perimeter edge 70, in similar fashion to the proximal end 38, distal end 39, and perimeter edge 40 of the aperture 37 of the cannula 30. The perimeter edge 70 of the sleeve 60 aperture 67 is configured to present a smooth, unsharpened edge to discourage shearing, tearing or cutting of the target visceral fatty tissue. In some embodiments, the aperture 67 of the sleeve 60 can be configured to prevent contact between tissue and any edges 40 of the aperture 37 of the cannula 30. For example, aperture 67 of the sleeve 60 is preferably smaller than aperture 37 of the cannula 30. In this manner, the edges 70 of the sleeve aperture 67 can effectively provide relief surfaces for the aperture 37 of the cannula 30. Accordingly, in such embodiments, the cannula body 59 can be constructed of a hard material, such as stainless steel, and the sleeve 60 can be configured to prevent contact between edges 40 and target visceral fat and non-target tissue.

The sleeve 60 is preferably configured to be disposable, such that the sleeve 60 can be removed from the cannula 30 and replaced with a new sleeve 60 between uses. In other embodiments, the sleeve 60 can be configured to be detachable and re-attachable to the cannula 30 for sterilization between uses. In either of these embodiments, the cannula 30 can be included in a kit that includes a plurality of sleeves 60, at least some of which can possess different characteristics, thereby allowing the surgeon to effectively adapt the cannula 30 according to specific surgical needs by selecting the appropriate sleeve 60. The sleeve 60 and the cannula 30 can define complimentary mating features for ensuring proper alignment of the apertures 37, 67 thereof. By way of one non-limiting example, the sleeve 60 can define an inwardly extending protrusion 72 configured to mate with, such as by being received within, a complimentary receptacle 74 defined in the outer surface 62 of the cannula 30. Other complimentary mating features are also within the scope of the present disclosure, such as ball-and-detent type mechanisms and the like. The complimentary mating features can also be configured to further retain the sleeve 60 is position relative to the cannula 30 during use.

The sleeve 60 is preferably constructed of an electrically insulative material, such as Teflon or other flexible polymeric materials like urethane, nylon, and others. In other embodiments, the sleeve 60 can be made of thin wall metallic materials like stainless steel and Nitinol in a non-continuous (coil or braid) to provide flexibility. The sleeve 60 material can be selected to avoid forming sharp edges when contacted against other objects, such as other surgical tools, cleaning instruments, and the like. Avoiding of the formation of sharp edges, such as burs, is important to prevent inadvertent cutting, abrading, scraping, or otherwise harming delicate non-target tissues within or adjacent the visceral fat being removed, such as organ tissue, vascular tissue, and mucosa, by way of non-limiting examples. The sleeve 60 material can also be selected such that an outer surface 76 of the sleeve 60 is smooth and has a low coefficient of friction (both static and sliding friction), thereby avoiding or at least reducing abrasion against delicate tissues within or adjacent visceral fat, such as those delicate tissues mentioned above. The sleeve 60 can also be subjected to one or more finishing processes to “smooth” or otherwise reduce the surface finish roughness (as measured by root square mean (RMS)) of the outer surface 76.

It should also be appreciated that, in other embodiments, the cannula 30 itself can be disposable. For example, any of the cannulas 30 disclosed herein can include a cannula body 59 that is constructed from a polymeric material that provides sufficient rigidity, such as polyetheretherketon (PEEK), polycarbonate (PC), high-density polyethylene (HDPE), nylon, and other high durometer plastics. In such embodiments, the cannula body 59 material can be selected to avoid forming sharp edges when contacted against other objects, such as other surgical tools, cleaning instruments, and the like. In such embodiments, a surgical kit can include a plurality of cannulas 30, each attachable to and non-destructively detachable from the handpiece 20.

In additional embodiments, the cannula body 59 can be constructed of a multi-layer polymer and metallic combination, such as a Teflon base inner body layer with a metallic braid or coil outer layer. In such embodiments, the cannula body 59 can be fitted with an outer sleeve 60, as described above. Such a multi-layer cannula 30 with sleeve 60 can be similar to existing coronary guiding catheter technologies like the one used in Cordis Vista Brite Tip Guiding Catheters and endoscopes like the one used in the Olympus Fiber Optic Lines (BF-XP60).

The cannula 30 can also include at least one cauterizing element 78, such as a cauterizing electrode, for mitigating bleeding that can result in some subjects during the visceral fat removal process. As shown in FIG. 6, the cauterizing electrode 78 can be a single tip electrode located at the distal end 32 of the cannula 30. In such embodiments, the distal end 64 of the sleeve 60 can be configured to define a distal opening that exposes the tip electrode 78. As shown in FIG. 7, in additional embodiments, the at least one cauterizing element 78 can include one or more ring electrodes 78 disposed along the outer surface 76 of the sleeve 60. In such embodiments, the sleeve 60 can extend over a distal end of the cannula body 59 and thereby the sleeve 60 can define the distal end 32 of the cannula 30. In embodiments where the sleeve 60 is omitted, the one or more ring electrodes 78 can be disposed along the outer surface 62 of the cannula body 59. In further embodiments, the cannula 30 can include a distal tip electrode 78 and one or more ring electrodes 78.

The at least one cauterizing element 78 is in electrical communication with a control element 80, such as a foot pedal or optionally a button or trigger located on the handpiece 20, by way of non-limiting examples. The control element 80 can be in electrical communication with the cauterizing electrode 78 via circuitry, such as one or more wires 82, which can extend through a channel or conduit, which can be enclosed and can be located within the cavity 90 of the cannula 30 or can be defined in the cannula body 59, or can extend between the outer surface 62 of the cannula 30 and an inner surface of the sleeve 60, by way of non-limiting examples. The physician can activate the at least one cauterizing electrode 78 as needed to cauterize or otherwise coagulate bleeding in the mesentery during the fat removal process.

Suitable dimensions for the cannula 30 outer diameter D2 range from 2.0 mm for small delicate visceral fatty deposits to 7.0 mm for large volume visceral fat removals as in an omentectomy. For open surgical procedures the entire range of cannula 30 sizes can be used, including outer diameters D2 up to about 20 mm. For laparoscopic or approaches requiring passage through other conduits or trocar sheaths, the cannula 30 outer diameter D2 range (i.e., measured at the outer surface 76 of the sleeve 60 or, in embodiments that omit the sleeve 60, measured at the outer surface 62 of the cannula body 59) should be from 2.0 mm to 7.0 mm, based on currently used abdominal trocar and access cannula sizes.

Suitable dimensions for the cannula 30 length L1 range from 2.0 cm for open surgical procedures to 50 cm for laparoscopic or procedures requiring access through other cannula sheaths. The cannula 30 preferably has between one (1) and six (6) apertures 37, which can be spaced on the same side of the cannula 30 or around the circumference of the cannula 30. More preferably, between one (1) and four (4) apertures 37 are used, all positioned on one side, which can be referred to as a “bottom side” or “active side” of the cannula 30. A preferred visceral cannula 30 has one (1), two (2), or three (3) apertures 37, all positioned on the bottom side so as to minimize pulling in non-target tissues and provide the surgeon with greater control.

The shapes of the apertures 37, 67 can be circular, elliptical, rectangular, triangular, or many other geometrical shapes. As shown in FIG. 8, the length L2 of the apertures 37, 67 is preferably greater than the width L3 thereof. Stated differently, the apertures 37, 67 are preferably elongated along the longitudinal direction L. One preferred shape for the apertures 37, 67 is an elliptical shape elongated along the longitudinal direction L. The size of the apertures 37, 67 can vary for any given cannula 30 diameter and length. The width L3 of the apertures 37, 67 is preferably smaller than half (50%) of the cannula 30 outer diameter D2. The length L2 of the apertures 37, 67 can be three quarters (75%) of the cannula diameter 30 or less. An effective cannula aperture 37 configuration is 1 mm in width L3 and 6 mm in length L2, according to one non-limiting example. Apertures 37, 67 configured in this manner (i.e., being longitudinally elongate) can provide increased fat removal efficiency when the surgeon moves the cannula 30 back and forth in a pivoting or arcing motion along the lateral direction (i.e., a motion analogous to a windshield wiper). Such a range of motion can include a total articulation angle of 90 degrees (i.e., ±45 degrees to either side), but smaller or larger articulation angles can be implemented, e.g., up to a total of 180 degrees (i.e., ±90 degrees to either side).

In an example embodiment, the cannula 30 can define an outer diameter D2 of about 2.0 mm measured at the outer surface 76 of the sleeve 60, and the cannula 30 and sleeve 60 can define, in complimentary fashion, a single pair of aligned apertures 37, 67 each having a perimeter edge 40, 70 that is smooth and unsharpened to avoid shearing, tearing or cutting of the target visceral fatty tissue and also to minimize trauma to non-target tissues. The aligned apertures 37, 67 can define a longitudinal length L2 greater than their lateral width L3.

In additional embodiments, the distal portion of the cannula 30 can optionally have two (2) pairs of apertures 37, 67 disposed on opposite sides of the cannula 30, similar to the manner shown in FIG. 2. In further embodiments, the cannula 30 can have different numbers of pairs of apertures 37, 67, which can be positioned in different configurations along the distal portion of cannula 30. In such embodiments, the pairs of apertures 37, 67 are preferably located on the same side of the cannula 30, though the pairs of apertures 37, 67 can optionally be angularly offset about the central axis 33 of the cannula 30.

The apertures 37 are configured to allow fatty tissue to enter the apertures 37 in response to vacuum pressure within the cannula shaft created by vacuum supply 14. Apertures 37 include a proximal end 38, a distal end 39, and a perimeter edge 40. Apertures 37 can take a generally circular, oval or egg shape, diamond or polygonal shape, or an amorphous shape. In the illustrated embodiments, the apertures 37 are oval and are all the same size. In alternative embodiments, the dimensions or shape of the apertures 37 can vary within a single cannula, like the cannula shown in FIG. 4, in which the diameter of each aperture 37 decreases in succession from the distal end to the proximal end.

The cannula 30 can be made of round tubing or tubing with one or more flat surfaces. The distal end 32 of the cannula 30 can be integrally formed as a continuation of the cannula 30 shaft or it can be a two piece construction, with a metal or polymer tip affixed to the end of the shaft. Polymer tips can be advantageous because they are softer than metal. Examples of suitable materials for the tip include, but are not limited to nylon and high density polyethylene.

In further embodiments (not shown), one or more articulation joints can be incorporated into the cannula 30. Preferably, these articulation joints are provided proximal to the proximal-most orifice. One suitable way to implement these articulating embodiments is to replace a portion of the cannula from a point that is proximal of the proximal-most orifice with a flexible tubing, and then running that flexible tubing through a conventional articulation joint. In these embodiments, the flexible tubing should be selected or reinforced so as not to collapse under the vacuums expected to be encountered. Examples of suitable articulation joints can be found in U.S. Pat. Nos. 4,108,211 and 7,090,637, each of which is incorporated herein by reference as if set forth herein in its entirety. Other examples include the bending mechanisms used in endoscopes like the Olympus Fiber Optic Lines (BF-XP60) and in other articulating devices like the Medtronic Heart Wall Ablation Catheters (RF Conductr (MC) Series). Any suitable conventional control mechanism can be used to bend the articulation joints, depending on the articulation joint that is used. Examples include foot pedals and hand controls.

When articulation joints with bending mechanisms are incorporated, the operator is able to control the position of the distal end 32 of the cannula 30. For example, if an articulation joint with a single degree of freedom is used, the operator would be able to bend the joint back and forth, like a windshield wiper so as to move the distal end in an arc, as described above.

If an articulation joint with two perpendicular degrees of freedom is used, the operator would be able to bend the joint back and forth, and would also be able to move the distal end of the probe up and down in a direction that is perpendicular to the plane of the wiping motion. These embodiments permit manipulation of the cannula's distal end 32 in three-dimensional space, providing additional fine-tuned control of movement, which can be particularly desirable when removing visceral fat from around intestinal structures. A suitable range of motion in the up/down direction is 45 degrees of bending, but smaller or larger articulation angles can be implemented, e.g., 80 degrees of bending. Optionally, the bending can be controlled by a mechanized, motorized unit under direct control of the surgeon.

The visceral fatty tissue lipectomy of the present invention should be target tissue-specific so as to remove the visceral fat without damaging the surrounding organs and tissue.

To accomplish this, the temperature of the solution is preferably between 75 and 250 degrees F., more preferably between 75 and 190 degrees F., still more preferably between 100 and 140 degrees F., and most preferably about 115 degrees F.

The stream pressure is preferably between 300 and 2000 psi, more preferably between 600 and 1300 psi, still more preferably between 800 and 1100 psi, and most preferably about 900 psi.

The fluid should preferably be delivered in pulses, with a preferred pulse rate between 25 and 60 pulses per second, more preferably about 40 pulses per second.

The preferred duty cycle for these pulses is between about 30% and 80%, more preferably about 30% to minimize the amount of waste fluid that is generated.

The preferred rise rate for the pulses is between about 0.1 to 3.0 milliseconds.

The aspiration (vacuum) is preferably between ⅓ and 1 atmosphere, and more preferably between ⅓ and ¾ atmosphere.

In the most preferred embodiments for removing visceral fat, the temperature of the solution is between 100 and 140 degrees F., and it is delivered in pulses at a stream pressure between 800 and 1100 psi at a pulse rate between 25 and 60 pulses per second. This combination of parameters provides good tissue differentiation, so as to facilitate removal of the visceral fat without causing trauma to the delicate anatomic structures in the vicinity. It should be appreciated that the foregoing fluid parameters, and those described throughout this disclosure, apply to each pulse of fluid individually. Stated differently, one or more and up to each of the fluid parameters can vary between individual pulses while remaining within the disclosed range(s) for the associated parameter(s).

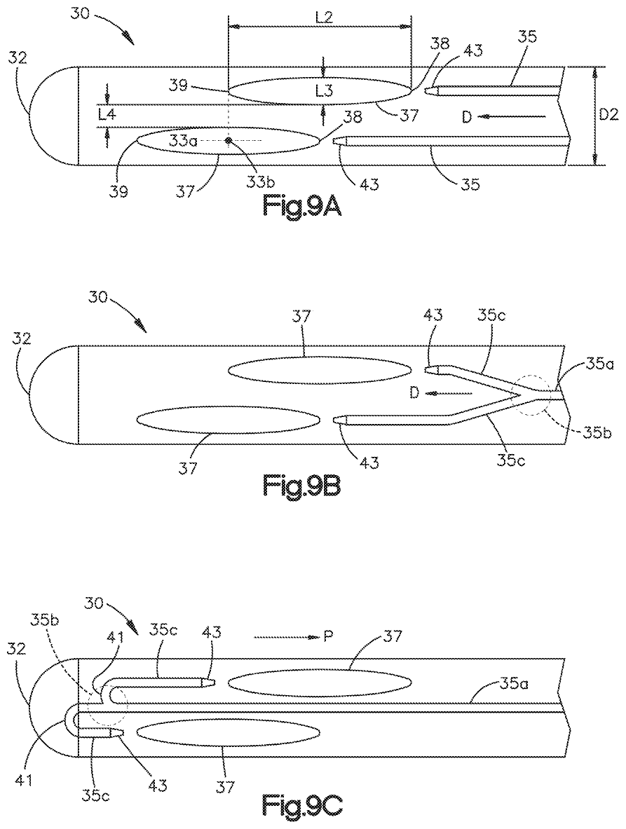

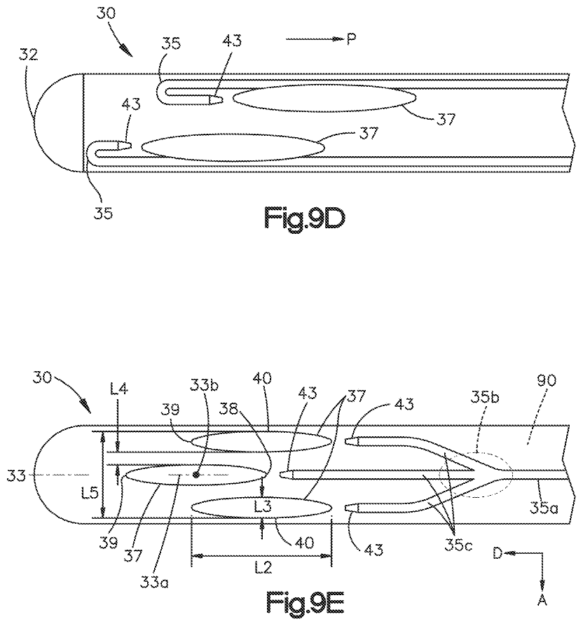

With reference to FIGS. 9A-9D, additional embodiments of cannulas 30 having two (2) laterally spaced (i.e., side-by-side) apertures 37 will now be described. In each of these embodiments, the cannula 30 includes two nozzles 43 that respectively direct boluses of fluid toward fatty tissue drawn into the inner cavity 90 through the apertures 37. In these embodiments, the apertures 37 are preferably the same size and shape, each being oblong and longitudinally elongate. For example, the apertures 37 can each have a length L2 that is about 6 times greater than their width L3, although other length-to-width ratios are within the scope of the present disclosure. The apertures 37 can also be spaced from each other by an offset distance L4, which can be measured along the circumference of the outer surface of the cannula 30. Alternatively, the offset distance L4 can be measured along the lateral direction A. The apertures 37 are also preferably longitudinally offset from each other or “staggered” such that the distal end 39 of the proximal-most one of the apertures 37 is longitudinally aligned with a longitudinal mid-point 33b (such as along the longitudinal aperture axis 33a) of the distal-most one of the apertures 37.

Referring now to FIG. 9A, the cannula 30 with side-by-side apertures 37 can include first and second supply tubes 35 with respective nozzles 43 that are proximally spaced from the proximal ends 38 of the apertures 37 and are configured to eject fluid boluses in the distal direction D. The first and second fluid supply tubes 35 can be connected to two separate pumps 19 having the same design. Stated differently, in this embodiment, each supply tube 35 has its own pump 19. Each pump 19 delivers pressurized, heated fluid to the supply tubes 35 so that series of boluses of the fluid are ejected from the respective nozzles 43.

As shown in FIG. 9B, a variation of the cannula 30 with side-by-side apertures 37 can include a single (1) main fluid supply tube 35a is connected to the pump 19. The main fluid supply tube 35a terminates distally at a junction 35b at which the main fluid supply tube 35a is bifurcated into a pair of supply tube branches 35c that have respective nozzles 43 that are proximally spaced from the proximal ends 38 of the apertures 37 and are configured to eject fluid boluses in the distal direction D.

As shown in FIG. 9C, another variation of the cannula 30 with side-by-side apertures 37 can include a single (1) main fluid supply tube 35a that terminates at a junction 35b, as above; however, in this variation the supply tube branches 35c define bends, such as U-bends 41 (or an alternative manifold as mentioned above), and include terminal portions that extend proximally to respective nozzles 43 that are distally spaced from the distal ends 39 of the respective apertures 37 and are configured to eject fluid boluses in the proximal direction P.

As shown in FIG. 9D, a further variation of the cannula 30 with side-by-side apertures 37 can include first and second fluid supply tubes 35 that each define a bend, such as a U-bend 41, that re-directs the fluid boluses in the proximal direction P. The first and second fluid supply tubes 35 have nozzles 43 at their terminal ends that are distally spaced from the distal ends 39 of the respective apertures 37 and are configured to eject fluid boluses in the proximal direction P. As above, the first and second fluid supply tubes 35 can be connected to two separate pumps 19 that are of the same design.

According to a non-limiting example of the present embodiments, the cannula 30 can define the following parameters: the cannula 30 defines an outer diameter D2 in a range of about 2.4 mm to about 3.5 mm; the apertures 37 each have a length L2 of about 6 mm and a width L3 of about 1 mm; the apertures 37 are spaced from each other by an offset distance L4 of about 0.5 mm (measured along the circumference of the cannula 30); the apertures 37 are longitudinally staggered such that the distal end 39 of the proximal-most aperture 37 is longitudinally aligned with a longitudinal mid-point 33b of the distal-most aperture 37; and the nozzles 43 each define an inner diameter that tapers to about 0.33 mm (about 0.013 inch).

The cannulas 30 according to the foregoing non-limiting example parameters can be employed with a pump system 19 that is configured to deliver boluses of larger volume (such as twice the volume) and similar pulse rates relative to the embodiments described above with reference to FIGS. 1-8. For example, for the cannula 30 variations shown in FIGS. 9B and 9C, the pump system 19 can employ a pump that is configured to deliver individual boluses each defining a volume of approximately 460-microliters. In such examples, approximately 460-microliter boluses travel down the single, main fluid supply tube 35a and then at the tube's bifurcation point (i.e., junction 35b) the boluses split in half resulting in the creation of approximately 230-microliter boluses which travel down each supply tube branch 35c; the end result is that approximately 230-microliter boluses are emerging from each of the supply tube nozzles 43. Thus, it can be said that the pump system 19 can deliver to the main fluid supply tube 35a a series of approximately 460-microliter boluses, which are divided at the junction 35b into respective first and second series of approximately 230-microliter boluses to the first and second supply tube branches 35c, which are then ejected from the respective nozzles 43. For the cannula 30 variations shown in FIGS. 9A and 9D, two pumps of the same design are utilized which deliver to each of the first and second fluid supply tubes 35 a series of boluses each having a volume of about approximately 230-microliters, which are ejected from the respective nozzles 43.

Alternatively, the cannulas 30 shown in FIGS. 9B-9C can be employed with a pump that delivers the boluses at a higher pulse rate (such as a pulse rate that is twice as great) and similar bolus volume relative to the embodiments described above with reference to FIGS. 1-8. For example, the pump can be configured to deliver boluses at a pulse rate of about 80 pulses per second and having individual bolus volumes of approximately 230-microliters.

Referring now to FIG. 9E, in further embodiments, the cannula 30 can have three (3) apertures 37. The apertures 37 are preferably the same size and shape, each being oblong and longitudinally elongate. For example, as above, the apertures 37 can each have a length L2 that is about 6 times greater than their width L3, although other length-to-width ratios are within the scope of the present disclosure. The apertures 37 can also be spaced from each other by an offset distance L4, which can be measured along the circumference of the outer surface of the cannula 30. Alternatively, the offset distance L4 can be measured along the lateral direction A. The apertures 37 can also be longitudinally staggered. For example, the three (3) apertures 37 can include a first, central aperture 37 that is distally offset from a second aperture 37 and a third aperture 37. The second and third apertures 37 can be longitudinally aligned with each other, optionally such that the distal ends 39 of the second and third apertures 37 are longitudinally aligned with a longitudinal mid-point 33b (such as along the longitudinal aperture axis 33a) of the first aperture 37. The apertures 37 can arranged so as to occupy a circumferential span L5 along the cannula 30, as measured about the longitudinal axis 33. The circumferential span L5 can be measured from opposite lateral-most edges 40 of the second and third apertures 37, and can be in a range of about 80 degrees to about 180 degrees, and more particularly in a range of about 120 degrees to about 160 degrees, and preferably about 140 degrees. It should be appreciated that other aperture arrangements, including staggered and non-staggered arrangements, are within the scope of the present disclosure.

The three-aperture 37 cannula 30 preferably includes three (3) nozzles 43 that respectively direct boluses of fluid toward fatty tissue drawn into the inner cavity 90 through the apertures 37. For example, the cannula 30 can include a single (1) main fluid supply tube 35a that is connected to the pump 19. The main fluid supply tube 35a terminates distally at a junction 35b at which the main fluid supply tube 35a is trifurcated into a trio of supply tube branches 35c that have respective nozzles 43 that are proximally spaced from the proximal ends 38 of the apertures 37 and are configured to eject fluid boluses in the distal direction D. Alternatively, one or more and up to all of the supply tube branches 35c can define a bend, such as a U-bend, that re-directs the fluid boluses in the proximal direction P across the apertures 37. The three-aperture cannula 30 can be employed with a pump system 19 that is configured to deliver boluses of larger volume (such as three times (3×) the volume) and similar pulse rates relative to the embodiments described above with reference to FIGS. 1-8. For example, the pump system 19 can employ a pump that is configured to deliver individual boluses each defining a volume of approximately 690 microliters to the main fluid supply tube 35a. In such examples, approximately 690-microliter boluses travel down the single, main fluid supply tube 35a and then split at the junction 35b into substantially equal thirds, resulting in the creation of approximately 230-microliter boluses which travel down each supply tube branch 35c; the end result is that approximately 230-microliter boluses are emerging from each of the supply tube nozzles 43. Alternatively, the three-aperture cannula 30 can be employed with a pump that delivers the boluses at a higher pulse rate (such as a pulse rate that is three times (3×) as great) and similar bolus volume relative to the embodiments described above with reference to FIGS. 1-8. For example, the pump can be configured to deliver boluses at a pulse rate of about 120 pulses per second and having individual bolus volumes of approximately 230-microliters. In other embodiments, a three-aperture 37 cannula 30 can include a trio of supply tubes each connected to a separate pump. For example, in such embodiments, each separate pump an deliver a respective series of boluses of approximately 230-microliters to the nozzles 43 for ejecting toward fatty tissue drawn into the cavity 90 through the apertures 37. Thus, the cannulas 30 of the foregoing multi-aperture embodiments should be matched with a pump configuration tailored to deliver boluses of desired volume from each nozzle 43.

It should be appreciated that the pump system 19 should utilize different pump configurations according to the number of apertures 37 provided on the cannula 30. For example, for a two-aperture cannula 30 having two fluid supply tubes 35 (FIGS. 9A and 9D), the pump system 19 employs two fixed-volume pumps each delivering boluses of about 230 microliters to the respective fluid supply tube 35. For a two-aperture cannula 30 having a single fluid supply tube 35 that is bifurcated into two supply tube branches 35c (FIGS. 9B and 9C), the pump system 19 employs a single, fixed-volume pump that delivers boluses of about 460 microliters to the supply tube 35. For a three-aperture cannula 30 having a single fluid supply tube 35 that is trifurcated into three supply tube branches 35c (FIG. 9E), the pump system 19 employs a single, fixed-volume pump that delivers boluses of about 690 microliters to the fluid supply tube 35. It should be appreciated that the tissue liquefaction system 2 can be configured to accommodate various pump systems 19, particularly those having pump configurations that are tailored for the specific number of apertures 37 provided by the cannula 30 (e.g., single pump, two pumps, three pumps). For example, the tissue liquefaction system 2 can be configured to interchangeably employ various fluid delivery sub-systems 3 each having an aperture-specific pump system 19 configurations. In such embodiments, the tissue liquefaction system 2 can include a fluid delivery kit that includes various fluid delivery sub-systems 3 that have different, aperture-specific pump systems 19. In other embodiments, the tissue liquefaction system 2 can employ a single fluid delivery sub-system 3 that itself can house or otherwise accommodate any of the foregoing pump configurations (e.g. single pump, two pumps, three pumps) in interchangeable fashion. In such embodiments, the tissue liquefaction system 2 can include a pump kit that includes various aperture-specific pump systems 19.

The multi-aperture 37 cannulas 30 described above provide significant advantages over prior art cannulas. In particular, the inclusion of additional apertures 37, such as second and/or third apertures 37, can significantly increase the rate of fat extraction by increasing the area through which fat is drawn into the interior cavity 90. Additionally, by providing the heated fluid from the fluid delivery sub-system 3 at the same functioning characteristics (e.g., temperature, pressure, bolus volume, pulse rate, rise rate) to each nozzle 43 (and thus across each aperture 37 to the fat drawn into the interior cavity 90), the fat extraction rate is further increased, especially relative to single-aperture 37 cannulas. Thus, the multi- aperture 37 cannulas 30 disclosed herein can significantly reduce the time necessary to extract visceral fat, thereby significantly reducing procedure duration.

It should be appreciated that the cannulas 30 constructed according to the embodiments of FIGS. 9A-9E can be employed with a sleeve 60 having apertures 67 that are aligned with the apertures 37 of the cannula 30, similar to the manner described above with reference to FIGS. 6-8. It should also be appreciated that the cannulas 30 constructed according to the embodiments of FIGS. 9A-9E can be employed with one or more cauterizing elements 78, similar to the manner described above.

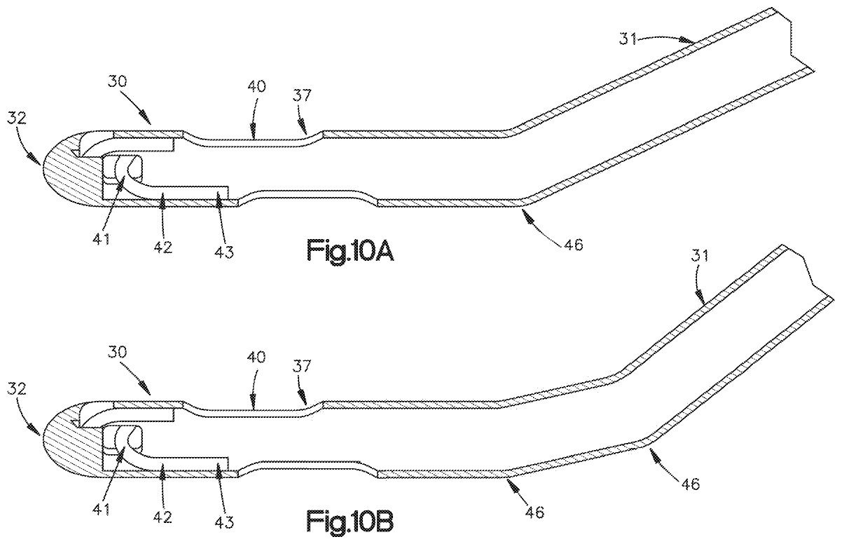

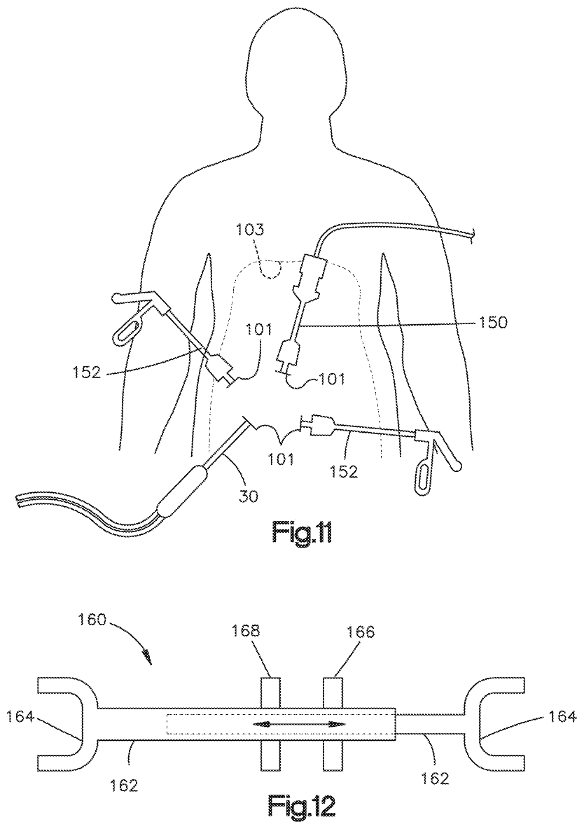

In further embodiments, the cannula 30 can define one or more bends, as shown in FIGS. 10A and 10B. In such embodiments, the distal-most bend 46 is preferably proximal to the proximal-most aperture 37. The bends 46 can be configured to aid the surgeon in the insertion and manipulation of the cannula 30, to conform to the particular anatomic region being treated. It should be appreciated that more than two (2) bends can be employed. The bends 46 can either be abrupt, such as by being defined by intersecting linear portions of the cannula (e.g., bent like a hockey stick), as illustrated in FIGS. 10A and 10B, or can involve radiused portions of the cannula that that define a radius of curvature, depending on the anatomic region being treated. The bends 46 are preferably proximal to all the apertures 37. Each bend 46 can define abend angle between 5 degrees and 85 degrees, as described more fully in the '700 Reference.

Visceral fat removal can be done using an open surgical methodology or a laparoscopic one. In an open methodology the cannula 30 is either inserted into the abdomen, or it is inserted into intra-abdominal anatomical tissues that have been exteriorized outside the abdominal cavity. In a laparoscopic approach, the visceral fat is visualized by a laparoscopic camera, as described in more detail below.

In an example method of an open procedure for removing visceral fat from a patient, the surgeon can make an incision, such as in the abdomen. The incision can be a vertical incision (i.e., an incision along the cranial-caudal direction) substantially down the center or slightly off-center of the abdomen. The incision penetrates the subcutaneous layer, the abdominal muscles, and the parietal peritoneum, providing access to the peritoneal cavity, particularly the mesentery. Retractors can be applied to the incised tissue to widen and maintain an open working channel providing access to the mesentery. The surgeon can insert the cannula 30 through the working channel and engage target visceral fat in the mesentery, including targeted fat in the omentum (e.g., greater omentum and lesser omentum), transverse mesocolon, small bowel mesentery, and sigmoid mesentery, as needed, by way of non-limiting examples. The surgeon can thereby employ the cannula 30 to liquefy, loosen, and aspirate the target visceral fat (or at least a major portion thereof) as needed. During this process, the surgeon can optionally externalize at least a portion of the small intestine, repeatedly and sequentially, a process referred to in the art as “running the bowel.”

Additionally or alternatively, the surgeon can use the working channel in the abdomen to access the extraperitoneal space to remove visceral fat from the anterior pararenal space and/or the perirenal space, the latter of which contains the kidneys. Removing visceral fat from the perirenal space (e.g., the perirenal fat capsule that surrounds the kidneys) using the cannula 30 can be referred to as a perirenal visceral lipectomy. In such a procedure that employs the abdominal working channel to access the perirenal space, the surgeon can make a secondary incision in the anterior renal fascia and can insert the cannula 30 through this secondary incision. In other embodiments, the surgeon can employ a different surgical approach to the perirenal space, such as a lateral approach, including an antero-lateral, true lateral, or a posterior-lateral approach.

Referring now to FIG. 11, in an example method of a laparoscopic procedure for removing visceral fat from a patient, the surgeon can make a series of incisions 101 in the patient's abdomen, including a first incision 101, such as for insertion of a hollow needle for inflating the peritoneal cavity 103, particularly the abdomen. Thereafter, the surgeon can remove the hollow needle and insert a laparoscope 150 in the first incision 101. The surgeon makes a second incision 101 in the abdomen and inserts the distal portion of the cannula 30 through the second incision 101 and into the inflated abdomen. The surgeon can make a third incision 101 in the abdomen for introduction of one or more additional surgical tools into the inflated abdomen, such as graspers 152 and the like for manipulating organs or other peritoneal anatomy as needed to allow the cannula 30 to access and engage visceral fat while avoiding, minimizing, or at least reducing contact between the cannula 30 and delicate non-target tissue. It should be appreciated that other instruments can also be inserted into the abdomen through the third incision, such as irrigation tubes for delivering surgical field cleaning fluids to the treatment area as needed, additional camera(s) or visualization instruments, diagnostic instruments, cauterizing and/or suturing tools, and the like. It should also be appreciated that the surgeon can make one or more additional incisions 101 in the abdomen as needed, such as for insertion of additional instrumentation.

With the laparoscope 150 and the cannula 30 inserted within the abdomen, the surgeon can manipulate the cannula 30 via the handpiece 20 to target and engage and thereby liquefy, loosen, and aspirate the target visceral fat (or at least a major portion thereof) as needed. As described above, the surgeon can control manipulation tools, such as graspers 152 and the like, to facilitate the targeting and engaging of visceral fat with the cannula 30.

In further examples, the surgeon can insert a first grasper 152 through a first respective one of the laparoscopic incisions 101 and can insert a second grasper 152 through a second respective one of the laparoscopic incisions 101. The surgeon can use the first and second graspers 152 to manipulate anatomy within the mesentery as needed to enhance access to visceral fat for the cannula 30. Referring now to FIG. 12, the surgeon can also employ a positioning tool 160 to control the relative position of the first and second grasper 152 with respect to each other. The positioning tool 160 can include pair of arms 162 that connect to the first and second graspers 152. It should be appreciated that the positioning tool 160 is meant to be used externally to the body, not inside the body. Its purpose is to enable the graspers 152 to hold the small bowel mesentery segment in a single plane, which facilitates safe extraction of mesenteric fat. For example, by using the graspers 152 to hold the small bowel mesentery segment in a single plane, the surgeon's hands are free to manipulate instrumentation to liquefy and aspirate visceral fat. Holding that particular segment in one plane allows the surgeon favorable access to the surgical site and favorable control during the fat removal process. Once that segment is completed, the positioning tool 160 is unlocked, the graspers 150 are re-positioned to the adjacent segment of the small bowel mesentery and fat removal in that segment is done, and that process can be repeated over and over through the length of the small bowel from distal ileum to proximal jejunum while avoiding the area of the root of the mesentery which is in the area of the proximal jejunum. In particular, the pair of arms can include a first arm 162 having a first receptacle 164 for coupling to the first grasper 152, and a second arm 162 having a second receptacle 164 for coupling to the second grasper 152. The positioning tool 160 can also include a locking mechanism 166 configured to iterate between a locked configuration, in which the relative position between the first and second arms 162 are affixed (thereby also affixing the relative position between the first and second graspers 152), and an adjustable configuration, in which the relative position between the first and second arms 162 (and thus also between the first and second graspers 152) is adjustable. The positioning tool 160 can further include an adjustment mechanism 168 that can be actuated to adjust the relative position between the first and second arms 162 (and thus also between the first and second graspers 152) in a controlled manner, as needed.

In an example method of an alternative laparoscopic technique for removing visceral fat from a patient, the surgeon can make a plurality of laparoscopic incisions in the abdomen, similar to the manner described above. The surgeon can then target and engage a localized segment of the small bowel mesentery with the cannula 30 to thereby liquefy, loosen, and aspirate the target visceral fat (or at least a major portion thereof) starting at the area of the distal ileum and then progressing in a sequential fashion, segment by segment, traveling in a generally cranial direction (i.e., towards the stomach). In that manner, the surgeon can effectively “run the bowel” laparoscopically inside the inflated abdomen with the one or more graspers (preferably two) to enable removal of visceral fat.

In each of the procedures described above, the visceral fat is preferably removed using a slow, controlled, methodical and precise movement of the cannula 30. The surgeon can engage visceral fat by moving the cannula 30 back and forth in the distal and proximal directions D, P. The surgeon can also move the cannula 30 in side-to-side motions along the lateral direction. In any of the procedures described above, the surgeon can also pivot the cannula 30 back and forth using a motion analogous to a windshield wiper. During testing, the inventors observed that such windshield wiper motions were particularly effective at increasing the efficiency of visceral fat removal without increasing potentially harmful contact against delicate non-target tissue. Such gains in efficiency were particularly observed in embodiments where the cannula 30 employs one or more apertures 37 having a greater total longitudinal orifice length than total lateral orifice length.

In any of the foregoing procedures, the surgeon can employ the cauterizing electrode(s) 78 as needed to mitigate any bleeding that might occur. The cauterizing electrode(s) 78 are particularly helpful because the allow the surgeon to cauterize bleeding as it occurs.

Optionally, surgical field cleaning fluids such as water or saline can be delivered to the site being treated during any of the foregoing procedures. Alternatively or in addition, a small amount of liposuction tumescent fluid (containing, e.g., epinephrine, lidocaine, levorphonal, phenylephrine, athyl-andrianol, ephedrine, or other vasoconstrictors and/or xylocalne, marcaine, nesacaine, Novocain, diprivan, ketalar, ladocaine, or other anesthetic agents and/or other suitable chemicals) can be introduced into the region that is being treated. One suitable way to introduce such fluids into the desired region is to include a dedicated conduit that is built into the cannula, such as with a distal-facing exit port similar, as described more fully in the '700 Reference. In alternative embodiments, a separate irrigation catheter can be used to introduce the desired fluids. These infusions are preferably implemented using low pressure peristaltic type pumping system, at a pressure less than 200 psi, and at an infusion flow rate between 50 and 600 ml min.

Minor bleeding was observed in some test subjects treated with the cannula 30. In these subjects, the cauterizing electrode(s) 78 was shown to quickly and successfully mitigate bleeding. It should be appreciated that other techniques can be employed to control bleeding, including techniques known in the surgical art of laparoscopic or open surgery.

It should be appreciated that any of the surgical techniques described above can be used with robotic assistance.

The embodiments described herein, particularly those for removing visceral fat, can be employed for treating one or more medical conditions in a subject, such that the subject experiences at least a reduction of symptoms of the medical condition. Examples of such medical conditions include metabolic syndrome (MS) (also referred to as “insulin resistance syndrome”), hypertension, cardiovascular disease, type II diabetes mellitus, obesity, Alzheimer's disease, dementia, cancer, aging, non-alcoholic fatty liver disease, and non-alcoholic steatohepatitis. For example, the embodiments that are optimized for removing visceral fat can be used during gastric bypass surgeries, or as stand-alone procedures for overweight or obese patients who do not qualify for gastric bypass surgery, especially for individuals with poorly controlled diabetes mellitus type II. Additionally, visceral fatty tissue lipectomy described in connection with these embodiments can be performed as a medical procedure for the prevention of diabetes mellitus type II and cardiovascular disease in patients with significant stores of visceral fat.

It should be appreciated that the embodiments described above can also be used in various liposuction procedures including, without limitation, liposuction of the face, neck, jowls, eyelids, posterior neck (buffalo hump), back, shoulders, arms, triceps, biceps, forearms, hands, chest, breasts, abdomen, abdominal etching and sculpting, flanks, love handles, lower back, buttocks, banana roll, hips, saddle bags, anterior and posterior thighs, inner thighs, mons pubis, vulva, knees, calves, shin, pretibial area, ankles and feet. They can also be used in revisional liposuction surgery to precisely remove residual fatty tissues after previous liposuction.

The embodiments described above can also be used in conjunction with other plastic surgery procedures in which skin, fat, fascia and/or muscle flaps are elevated and/or removed as part of the surgical procedure. This would include, but is not limited to facelift surgery (rhytidectomy) with neck sculpting and submental fat removal, jowl excision, and cheek fat manipulation, eyelid surgery (blepharoplasty), brow surgery, breast reduction, breast lift, breast augmentation, breast reconstruction, abdominoplasty, body contouring, body lifts, thigh lifts, buttock lifts, arm lifts (brachioplasty), as well as general reconstructive surgery of the head, neck, breast abdomen and extremities. It will be further appreciated that the embodiments described above have numerous applications outside the field of liposuction.

The embodiments described above can be used in skin resurfacing of areas of the body with evidence of skin aging including but not limited to sun damage (actinic changes), wrinkle lines, smokers' lines, laugh lines, hyper pigmentation, melasma, acne scars, previous surgical scars, keratoses, as well as other skin proliferative disorders.

The embodiments described above can target additional tissue types including, without limitation, damaged skin with thickened outer layers of the skin (keratin) and thinning of the dermal components (collagen, elastin, hyaluronic acid) creating abnormal, aged skin. The cannula would extract, remove, and target the damaged outer layers, leaving behind the healthy deep layers (a process similar to traditional dermabrasion, chemical peels (trichloroacetic acid, phenol, croton oil, salicyclic acid, etc.) and ablative laser resurfacing (carbon dioxide, erbium, etc.) The heated stream would allow for deep tissue stimulation, lightening as well as collagen deposition creating tighter skin, with improvement of overall skin texture and/or skin tone with improvements in color variations. This process would offer increased precision with decreased collateral damage over traditional methods utilizing settings and delivery fluids which are selective to only the damaged target tissue.

Other implementations include various distal tip designs and lighter pressure settings that can be used for tissue cleansing particularly in the face but also applied to the neck, chest and body for deep cleaning, exfoliation and overall skin hydration and miniaturization. Higher pressure settings can also be used for areas of hyperkeratosis, callus formation in the feet, hands knees, and elbows to soften, hydrate and moisturize excessively dry areas.

Additional tissue removal procedures can be accomplished by various other embodiments. For example, viable fat cells (adipocytes) can be extracted and processed for re-injection into areas of fat deficiency. This would include, without limitation, areas around the face, brow, eyelids, tear troughs, smile lines, nasolabial folds, labiomental folds, cheeks, jaw line, chin, breast, chest abdomen, buttocks, arms, biceps, triceps, forearms, hands, flanks, hips, thighs, knees, calves, shin, feet, and back. A similar method can be used to address post liposuction depressions and/or concavities from over aggressive liposuction. Other procedures utilizing a similar method include; without limitation, breast augmentation, breast lifts, breast reconstruction, general plastic surgery reconstruction, facial reconstruction, reconstruction of the trunk and/or extremities.

Additional uses include tissue removal in the spine or spinal nucleotomy. The cannula used in spinal nucleotomy procedures includes heated solution supply tubes within the cannula as described above. The cannula further includes a flexible tip capable of moving in multiple axes, for example, up, down, right and left. Because of the flexible tip, a surgeon can insert a cannula through an opening in the annulus fibrosis and into the central area, where the nucleus pulpous tissue is located. The surgeon can then direct the cannula tip in any direction. Using the cannula in this manner the surgeon is able to clean out the nucleus pulpous tissue while leaving the annulus fibrosis and nerve tissue intact and unharmed.

In another implementation, the present design can be incorporated in to an endovascular catheter for removal of vascular thrombus and atheromatous plaque, including vulnerable plaque in the coronary arteries and other vasculature.

In another implementation, a cannula using the present design can be used in urologic applications that include, but are not limited to, trans-urethral prostatectomy and trans-urethral resection of bladder tumors.

In another implementation, the present design can be incorporated into a device or cannula used in endoscopic surgery. An example of one such application is chondral or cartilage resurfacing in arthroscopic surgery. The cannula can be used to remove irregular, damaged, or torn cartilage, scar tissue and other debris or deposits to generate a smoother articular surface. Another example is in gynecologic surgery and the endoscopic removal of endometrial tissue in proximity to the ovary, fallopian tubes or in the peritoneal or retroperitoneal cavities.

In yet a further implementation to treat chronic bronchitis and emphysema (COPD), the cannula can be modified to be used in the manner a bronchoscope is used; the inflamed lining of the bronchial tubes would be liquefied and aspirated, thereby allowing new, healthy bronchial tube tissue to take its place.

The various embodiments described each provide at least one of the following advantages: (1) differentiation between target fatty tissue and delicate non-target tissue to an extent allowing clinical removal of visceral fat, which to the inventors' knowledge had not been possible without significant risk to the health of the patient; (2) a reduction in the level of suction compared to traditional liposuction, which mitigates damage to non-target tissue; (3) a significant reduction in the time of the procedure and the amount of cannula manipulation required; (4) a significant reduction in surgeon fatigue; (5) a reduction in blood loss to the patient; and (6) improved patient recovery time because shearing of tissue is not the enabling mechanism of fat removal, liquefaction is instead; shearing and suction of sheared tissue pieces is replaced with liquefaction and suction of liquefactant.

Although the present invention has been described in detail with reference to certain implementations, other implementations are possible and contemplated herein. All the features disclosed in this specification can be replaced by alternative features serving the same, equivalent, or similar purpose, unless expressly stated otherwise. Thus, unless expressly stated otherwise, each feature disclosed is one example only of a generic series of equivalent or similar features.

Furthermore, it should be understood that various changes, substitutions, and alterations can be made herein without departing from the spirit and scope of the invention as defined by the appended claims. For example, features of the various embodiments described herein can be incorporated into one or more and up to all of the other embodiments described herein. Moreover, the scope of the present disclosure is not intended to be limited to the particular embodiments described in the specification. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein can be utilized according to the present disclosure.

What is claimed:

1. A method of removing visceral fat from a subject, the method comprising:
inserting at least a distal portion of a cannula into anatomy of the subject, wherein the anatomy comprises a peritoneal cavity, a retroperitoneal cavity, or visceral fat that has been repositioned from its natural anatomical location in the body of the subject, wherein the cannula is elongate along a longitudinal direction and the distal portion defines at least one aperture that is open to an interior cavity of the cannula;
generating a negative pressure in an interior of the cannula, thereby drawing a portion of visceral fat from the anatomy, through the at least one aperture and into the interior cavity;
delivering fluid through a conduit of the cannula along the longitudinal direction, wherein the fluid is delivered as a series of pulses that are individually at a pressure in a range of 600 psi to 1300 psi and at a temperature in a range of 80 degrees Fahrenheit and 140 degrees Fahrenheit;
expelling the fluid in the series of pulses from the conduit, wherein each expelled pulse of the fluid comprises a bolus having a volume between 215 microliters and 245 microliters;
impacting the expelled fluid against the portion of visceral fat so as to liquefy the visceral fat; and
suctioning at least a major portion of the liquefied visceral fat through the interior of the cannula and away from the subject responsive to the negative pressure.

2. The method of claim 1, wherein:
the fluid is delivered at a temperature of about 115 degrees Fahrenheit;
each bolus is delivered at a pressure between 600 and 1300 psi; and
the fluid is delivered at a pulse rate in a range of 25 to 60 pulses per second.

3. The method of claim 2, wherein the pulses are delivered with a duty cycle in a range of 30 percent to 80 percent, the fluid boluses have a rise rate of between 0.1 and 3.0 milliseconds, and the pressure is in a range of 800 psi to 1100 psi.

4. The method of claim 1, further comprising making an incision through the abdomen and opening the incision, wherein the inserting step comprises inserting the distal portion of the cannula through the open incision, into the peritoneal cavity, and into the mesenteric visceral fat of the subject.

5. The method of claim 1, further comprising, after the inserting step:
moving the distal portion of the cannula back and forth in an arc motion along an arc direction that is substantially perpendicular to the longitudinal direction; and
moving the distal portion of the cannula back and forth along the longitudinal direction while moving the distal portion back and forth in the arc motion.

6. The method of claim 1, further comprising:
making a series of laparoscopic incisions in the abdomen of the patient;
delivering gas into the abdomen through at least one of the laparoscopic incisions, thereby inflating the peritoneal cavity; and
inserting a laparoscope through one of the laparoscopic incisions;
wherein the step of inserting the distal portion of the cannula comprises inserting the distal portion into the mesentery of the peritoneal cavity through another one of the laparoscopic incisions.

7. The method of claim 6, further comprising:
inserting a first grasper through a first respective one of the laparoscopic incisions and inserting a second grasper through a second respective one of the laparoscopic incisions;
manipulating the first and second graspers to reposition at least one anatomical structure within the mesentery to access visceral fat with the cannula;
after the manipulating step, engaging the first and second graspers with a positioning tool, wherein the positioning tool engages the first and second graspers outside of the body; and
iterating the positioning tool between:
a locked configuration in which a relative position between the first and second graspers is affixed, and
an adjustable configuration in which the relative position between the first and second graspers is adjustable.

8. The method of claim 1, further comprising treating a medical condition in a subject
such that the subject experiences at least a reduction of symptoms of the medical condition, and the medical condition is selected from the group comprising metabolic syndrome, hypertension, cardiovascular disease, type II diabetes mellitus, obesity, Alzheimer's disease, dementia, cancer, aging, non-alcoholic fatty liver disease, and non-alcoholic steatohepatitis.
